(19) United States
(12) Patent Application Publication
(10) Pub. No.: US 2026/0341472 A1
(43) Pub. Date: Oct. 29, 2026

(54) MEDICAL IMAGING SYSTEMS AND METHODS FOR CHARACTERIZING TISSUE

(71) Applicant: Boston Scientific Scimed, Inc., Maple Grove, MN (US)

(72) Inventors: Michael Francis McGovern, San Diego, CA (US); Veerakumar Venugopal, Carver, MN (US); Longquan Chen, Lexington, MA (US); Zhuolin Wu, Circle Pines, MN (US)

(73) Assignee: Boston Scientific Scimed, Inc., Maple Grove, MN (US)

(21) Appl. No.: 19/657,467

(22) Filed: Apr. 24, 2026

Related U.S. Application Data
(60) Provisional application No. 63/795,859, filed on Apr. 28, 2025.

(51) Int. Cl. G16H 30/20

(52) U.S. Cl. CPC G16H 30/20

(57) ABSTRACT

A method for processing endoscopic image data, including receiving, by a controller, endoscopic image data of a subject during an endoscopic procedure from an endoscopic device coupled to the controller by a connector; preprocessing, by a module housed in the controller, the endoscopic image data; providing the preprocessed endoscopic image data to a trained machine learning model stored on the controller; using the trained machine learning model to characterize a tissue of the subject during the endoscopic procedure; and displaying a user interface that communicates a characterization of the tissue of the subject.

### PRIORITY

This application claims the benefit of priority under 35 USC § 119 to United States Provisional Patent Application Serial No. 63/795,859, filed Apr. 28, 2025, which is incorporated by reference herein in its entirety and for all purposes.

### TECHNICAL FIELD

The disclosure relates generally to systems and methods for medical image processing. More specifically, aspects of the disclosure pertain to systems and methods for medical image processing to characterize tissue.

### BACKGROUND

A medical system may include a medical device, such as an endoscope, that is removably connectable to a computing system to perform a medical procedure. For example, the endoscope may be inserted into and navigated through a lumen of a patient to a target site during a medical procedure. The endoscope may include an imaging device configured to capture images, as well as a light source configured to emit light to facilitate visualization of anatomical features and/or or other objects within the lumen that are captured by the images. The computing system may process the captured images received from the endoscope and display the processed images for viewing by an operator (e.g., a physician). During an endoscopic procedure, a user (e.g., a physician) may view and characterize tissue using the displayed images. However, operator skill levels may vary widely, and operators may fail to correctly characterize tissue (e.g., as malignant or benign).

### SUMMARY

This summary is provided to introduce a selection of concepts in a simplified form that are further described below in the detailed description.

In some aspects, the techniques described herein relate to a method for processing endoscopic image data, including: receiving, by a controller, endoscopic image data of a subject during an endoscopic procedure from an endoscopic device coupled to the controller by a connector; preprocessing, by a module housed in the controller, the endoscopic image data; providing the preprocessed endoscopic image data to a trained machine learning model stored on the controller; using the trained machine learning model to characterize a tissue of the subject during the endoscopic procedure; and displaying a user interface that communicates a characterization of the tissue of the subject.

The techniques or methods described herein may include any or all of the following features or steps in any combination.

The endoscopic image data includes video frames captured during the endoscopic procedure. The user interface is updated in real-time during the endoscopic procedure. Preprocessing the endoscopic image data includes image normalizing to standardize brightness and color values across different endoscopic images.

The trained machine learning model is generated using a methodology including pruning, quantizing, or sparsifying. The trained machine learning model includes a convolutional neural network.

Characterizing the tissue of the subject includes detecting a presence of a stricture. Characterizing the tissue of the subject further includes classifying the detected stricture. Characterizing the tissue of the subject includes determining a likelihood of malignancy for observed tissue features.

The user interface includes a confidence score for a tissue classification. The confidence score is overlaid on a live endoscopic image, and the confidence score is updated in real-time. The user interface includes a visual overlay on the endoscopic image data to highlight a region of interest.

The trained machine learning model is trained using pathology data. The trained machine learning model is trained by comparing the pathology data to annotated training image data.

The method further includes storing the characterization of the tissue for subsequent training of the machine learning model.

In some aspects, the techniques described herein relate to a method for training a machine learning model to characterize tissue from endoscopic images, including: receiving annotated image data obtained from endoscopic procedures performed on one or more subjects, wherein the annotated image data includes annotations characterizing tissue of the one or more subjects; using the received annotated image data to train a machine learning model to characterize tissue from endoscopic images; receiving pathology data that characterizes the tissue of the one or more subjects; comparing the annotated image data to the pathology data; and outputting a trained machine learning model based on comparing the annotated image data to the pathology data.

The techniques or methods described herein may include any or all of the following features or steps in any combination.

Comparing the annotated image data to the pathology data includes determining whether annotations of the annotated image data include errors. The pathology data and annotations of the annotated image data characterize the same tissue.

In some aspects, the techniques described herein relate to a method for training a machine learning model to characterize tissue from endoscopic images, including: receiving pathology data that characterizes a same tissue that was previously characterized by a trained machine learning model; comparing the received pathology data to a previous characterization of the same tissue that was made by the trained machine learning model; and based on the comparing, refining the trained machine learning model.

The techniques or methods described herein may include the following features.

Refining the trained machine learning model includes adjusting parameters of the machine learning model to improve alignment between the model's previous characterization of the tissue and the pathology data.

The foregoing general description of the illustrative embodiments and the following detailed description thereof are merely exemplary aspects of the teachings of the disclosure and are not restrictive.

It may be understood that both the foregoing general description and the following detailed description are exemplary and explanatory only and are not restrictive of the invention, as claimed. As used herein, the terms “comprises,” “comprising,” “includes,” “including,” “has,” “having,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.” The term “distal” refers to a direction away from an operator/toward a treatment site, and the term “proximal” refers to a direction toward an operator. The term “approximately,” or like terms (e.g., “substantially”), includes values +/- 10 % of a stated value.

### BRIEF DESCRIPTION OF FIGURES

Non-limiting and non-exhaustive examples are described with reference to the following figures. The accompanying drawings, which are incorporated in and constitute a part of this specification, illustrate examples of the disclosure and, together with the description, serve to explain the principles of the disclosure.

FIG. 1 depicts an example medical system, according to aspects of the disclosure.

FIG. 2 illustrates an exemplary block diagram of a processing system for analyzing video frames, according to aspects of the disclosure.

FIG. 3 illustrates an exemplary flowchart of a process for training a machine learning model, according to aspects of the disclosure.

FIG. 4 illustrates an exemplary block diagram of a model generation method, according to aspects of the disclosure.

FIG. 5 illustrates an exemplary flowchart depicting a process for processing endoscopic image data, according to an embodiment.

FIG. 6 illustrates a flowchart of a process for training a machine learning model using pathology data, according to aspects of the disclosure.

FIG. 7 illustrates a block diagram depicting example processes for training, using, and monitoring the model.

FIG. 8 illustrates an exemplary display output, according to aspects of the disclosure.

FIG. 9 illustrates a block diagram of a computing device, according to aspects of the disclosure.

### DETAILED DESCRIPTION

The following description sets forth exemplary aspects of the disclosure. It should be recognized, however, that such description is not intended as a limitation on the scope of the disclosure. Rather, the description also encompasses combinations and modifications to those exemplary aspects described herein.

A detailed description of systems, devices, and methods consistent with embodiments of the disclosure is provided below. While several embodiments are described, it should be understood that this disclosure is not limited to any one embodiment, but instead encompasses numerous alternatives, modifications, and equivalents. In addition, while numerous specific details are set forth in the following description in order to provide a thorough understanding of the embodiments disclosed herein, some embodiments can be practiced without some or all of these details. Moreover, for the purpose of clarity, certain technical material that is known in the related art has not been described in detail in order to avoid unnecessarily obscuring the disclosure.

Endoscopic retrograde cholangiopancreatography (ERCP) is a procedure that utilizes endoscopy to diagnose and/or treat conditions of the biliary and pancreatic systems, such as strictures, pancreatic and ampullary cancers, tumors, intraductal papillary mucinous neoplasm, or other conditions. Diagnosing and/or treating such conditions depends on the accurate identification and/or characterization of the strictures and/or tissues. Conventionally, Diagnosing and/or treating such conditions depends on the accurate identification and/or characterization of the strictures. Conventionally, an operator may identify and/or characterize a stricture or other tissue feature by viewing endoscopic images on a display. The accuracy and efficiency of such identification and/or characterization may vary significantly between operators. Incorrect identification and/or characterization of tissue (e.g., a stricture) may potentially lead to worse patient outcomes.

To address and overcome these challenges, systems and methods are described herein for identifying and characterizing strictures. For example, the disclosed systems and methods may characterize tissue (e.g., a stricture) in real-time during an endoscopic procedure, such as ERCP. Machine learning models, such as Convolutional Neural Networks (CNN), may be utilized in the disclosed systems and methods. In some aspects, the CNNs may include vision transformers (ViTs) or other neutral networks. The machine learning models may analyze endoscopic images or video frames to detect and classify strictures. For example, the model(s) may provide real-time feedback to the endoscopist. Although the term machine learning model is used herein, that the term also includes other types of models, including other types of artificial intelligence models.

The systems and methods described herein may offer several technical advantages. For example, they may provide more accurate stricture or lesion identification, thereby helping to improve diagnostic accuracy and treatment outcomes. In some aspects, they may provide more accurate stricture identification in the subject or more accurate identification of cancerous tissues that may cause pancreatic or ampullary cancer or intraductal papillary mucinous neoplasm. Real-time analysis may allow for immediate decision-making during the procedure, potentially reducing procedure time and improving patient comfort. Additionally, the use of machine learning algorithms may allow for the detection of subtle features or patterns that may not be immediately apparent to the human eye, potentially leading to earlier or more precise diagnoses.

The systems and methods described herein are primarily described and referred to throughout the following description for providing more accurate identification and characterization of strictures, cancerous tissues, or other lesions in the subject. However, the systems and methods may be utilized to identify and characterize other types of tissues, tumors, growths, foreign bodies, anatomical structures, and more. Although strictures may be referenced as an example use-case in the below description, the disclosed systems, devices, and methods may also apply to other conditions or structures, such as tissues, lesions, tumors, growths, foreign bodies, anatomical structures, etc.

In some aspects, the disclosed systems may be implemented on a controller unit to which a scope device is directly coupled. The disclosed systems may be implemented on a central processing unit (CPU) onboard such a controller, rather than offboard on a graphics processing unit (GPU). Such an implementation may reduce a need for a hospital or other medical institution to acquire additional equipment. The techniques disclosed herein may enable use of the disclosed systems on such a controller.

Furthermore, the disclosed systems and methods may use a wide range of images for training the disclosed machine learning models. These images may include those with clearly identifiable strictures or tissues, those with subtle or ambiguous stricture presentations or lesions, and those without any identifiable strictures or lesions at all. By incorporating this diverse spectrum of images, the machine learning models can learn to distinguish between normal anatomical structures and various types of strictures across different presentations, lighting conditions, and patient anatomies. As compared with a model trained on a more constrained image set, the disclosed machine learning model has more robust feature extraction capabilities and decision boundaries, resulting in significantly improved classification accuracy in real-world clinical scenarios where image quality and stricture presentation can vary substantially.

In some aspects, the systems and methods may incorporate multiple data sources, such as endoscopic images, fluoroscopy data, and patient history, to provide a more comprehensive analysis of the stricture or lesion. Data may be obtained from medical procedures using the trained machine learning models disclosed herein. This data may be used to improve the machine learning model. Furthermore, pathology data may be utilized to verify training data sets, thereby improving the machine learning model, as described in detail below. The pathology data serves as a ground truth reference against which the model's predictions can be systematically evaluated and validated. For example, by comparing the model's characterizations with definitive pathological findings from biopsies, surgical specimens, or other diagnostic procedures, the system can be used to improve the trained machine learning model’s performance. For example, the integration of pathology may create a continuous feedback loop that progressively enhances the model's accuracy in distinguishing between benign and malignant strictures, potentially reducing false positives and false negatives. Additionally, this pathology-based verification approach helps mitigate biases that might exist in the initial training data, ensuring the model performs consistently across diverse patient populations and varying presentations of strictures or tissues. In some aspects, the pathology-based verification approach may optimize the model to adapt to changes in the data. The pathology-based verification may provide for a closed-loop feedback scheme.

It should also be understood that the examples above are illustrative only. The techniques and technologies described herein may be adapted to various endoscopic procedures and types of tissue characterization.

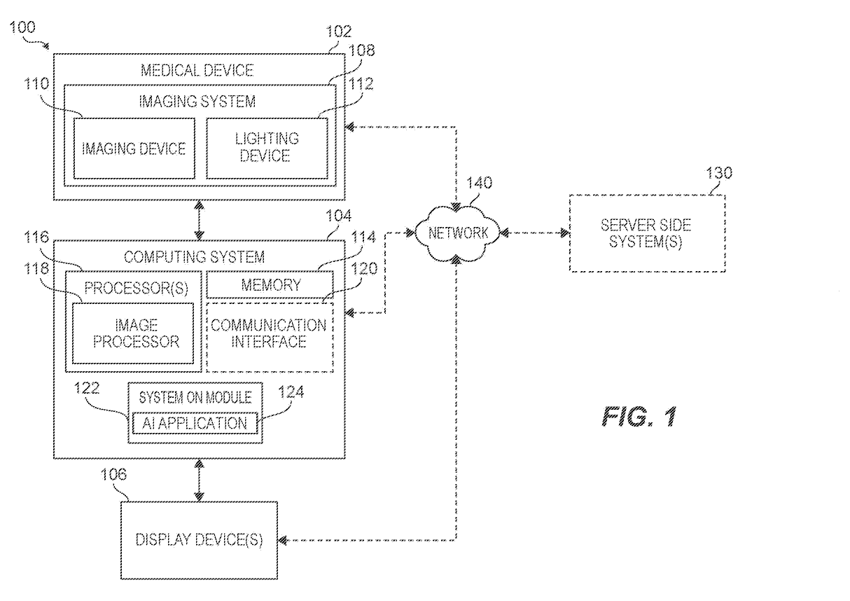

FIG. 1 depicts an example medical system 100 in which various processes disclosed herein may be implemented. Medical system 100 may include a medical device 102, a computing system 104, one or more display device(s) 106, one or more server-side system(s) 130, and/or a network 140.

Medical device 102 may be used to perform a diagnostic and/or interventional medical procedure on a patient upon connection with computing system 104. Medical device 102 may be an endoscope or other type of scope, such as a bronchoscope, ureteroscope, duodenoscope, cholangioscope, gastroscope, endoscopic ultrasonography ("EUS") scope, colonoscope, laparoscope, arthroscope, cystoscope, aspiration scope, sheath, or catheter, among other examples. For example, medical device 102 may particularly be a duodenoscope or a cholangioscope used during an ERCP procedure.

In some examples, medical device 102 may be a single use device that is discarded or disposed of upon disconnection from computing system 104 (e.g., at an end of the medical procedure). Therefore, although only one of medical device 102 is shown in FIG. 1, medical system 100 may include one or more other medical devices 102 that are the same or similar to medical device 102, and may also be removably connected to computing system 104 for performing medical procedures. Additionally, in some examples, different types of medical devices may be connectable to computing system 104 simultaneously with or independently from medical device 102.

Medical device 102 may be comprised of a handle, a shaft, and an umbilicus. At least a portion of the shaft, including a distal end of shaft, may be inserted into and navigated through a body lumen of a patient to a target site during a medical procedure, such that distal end is positioned proximate to the target site. Distal end may include distal openings for one or more of a working channel, an insufflation channel, or a fluid irrigation channel. Working channel may be a lumen that extends through the shaft and provides for delivery of instruments or treatment objects to the target site or removal of objects from the target site at distal end. Working channel may also provide suctioning functionality. The insufflation channel may be a lumen that extends through shaft and provides insufflation at distal end. The fluid irrigation channel may be a lumen that extends through the shaft and provides irrigation functionality at distal end to increase visualization, for example. In some examples, the insufflation and fluid irrigation channel may be a same channel or may exit out of a same distal opening. The distal end may also include one or more components of an imaging system 108.

Imaging system 108 may include at least one imaging device 110 and at least one lighting device 112. Imaging device 110 may be located at a distal end of medical device 102 (e.g., at a distal tip of medical device 102). Imaging device 110 may be configured to continuously capture image signals as the distal end of medical device 102 is inserted into and navigated through a body lumen of the patient to a target site during the medical procedure. Imaging device 110 may include one or more cameras, one or more image sensors, or one or more endoscopic viewing elements, among other similar devices. In some examples, lighting device 112 may be located at the distal end of medical device 102 (e.g., at the distal tip of medical device 102) along with imaging device 110. In other examples (not shown), lighting device 112 may be a separate device or may be integrated with computing system 104, with light from lighting device 112 being transmitted via fibers (e.g., optical fibers) extending a length of medical device 102 (e.g., from a proximal end of medical device 102 connected to computing system 104 to the distal tip of medical device 102). Lighting device 112 may be configured to illuminate areas of the patient's body (e.g., the target area) during the medical procedure to facilitate imaging of the target area by imaging device 110. Lighting device 112 may include one or more LEDs, incandescent light sources, optical fibers (e.g., optical fibers to transmit a light from a proximal light source), or other illuminators.

The handle may include one or more actuators. The actuators may provide control over a steerable section at the distal portion of the shaft (e.g., having an articulation joint), an elevator, or imaging functions at the distal end of shaft, as well as allow for the provision of air, water, suction, etc. For example, the handle may include control knobs for left, right, up, or down control of the steerable section of shaft. For example, one of control knobs may provide left/right control of the steerable section, and the other of control knobs may provide up/down control of the steerable section. The handle may further include one or more locking mechanisms (e.g., knobs or levers) for preventing steering or braking of the steerable section in at least one of an up, down, left, or right direction. The handle may include an elevator control lever (e.g., if medical device is a duodenoscope or an endoscopic ultrasound scope). The elevator control lever may raise or lower an elevator, via a connection between the lever and an actuating wire that extends from the lever, through shaft, to the elevator.

The umbilicus connects the handle, the shaft, the distal end, and the components at the distal end (e.g., imaging system 108) to one or more sources of, for example, power, imaging device control, image processing, light, light control, or display equipment. As one example, the umbilicus connects to computing system 104 (e.g., via a connector plug), and supports the transmission of various data signals between computing system 104 and medical device 102. For example, the umbilicus may support the transmission of data between medical device 102 and computing system 104 to facilitate communication establishment and set-up of operating parameters. Additionally, the umbilicus may support the transmission of power and control signals from computing system 104 to medical device 102, and particularly to imaging system 108. Further, the umbilicus may support the transmission of image data from imaging system 108 to computing system 104 for processing and display via display device(s) 106.

One or more components of medical device 102, including imaging system 108 and the components thereof, may be communicatively coupled to computing system 104 via wired connections or wireless connections (e.g., over network 140) to enable communication of various signals between medical device 102 and computing system 104. For example, image signals captured by imaging device 110 (e.g., raw image data) may be received by computing system 104. Additionally, computing system 104 may provide one or more signals to the imaging device 110 or lighting device 112 to cause one or more parameters of imaging device 110 or lighting device 112, respectively, to be adjusted.

In some examples, computing system 104 is a controller, a control unit, a computing device, or other similar standalone processing unit separate from medical device 102. In other examples, computing system 104 may be partially or fully integrated with medical device 102. For example, computing system 104 may be partially or fully positioned in the handle of medical device 102. As another example, computing system 104 may be partially or fully positioned at the distal end of medical device 102. Computing system 104 may be or at least include a field-programmable gate array (FPGA) comprising programmable logic blocks for performing various functions.

Computing system 104 may include at least one memory 114, one or more processor(s) 116, and at least one communication interface 120. In some examples, memory 114, processor(s) 116, and communication interface 120, may be embedded on a printed circuit board (PCB) forming a system-on-module (SOM) of computing system 104. Memory 114 may store instructions to be executed by processor(s) 116 to cause computing system 104 to perform corresponding operations. At least a portion of the instructions stored in memory 114 may include instructions for characterizing tissue. In some aspects, memory 114 may be used to store machine learning models, such as CNNs, AI models, or aspects of models (e.g., weights). Memory 114 may also store any models, algorithms, or other processes that are executable to perform the operations, such as one or a combination of the SfM or SLAM algorithms. Memory 114 may include one or more data stores for such storage. Additionally or alternatively, computing system 104 may include one or more data stores separate from memory 114 for such storage.

Processor(s) 116 may include at least one image processor 118. Image processor 118 may be configured to process one or more image signals (e.g., raw image data) captured by imaging device 110 and received by computing system 104 to generate an image. Image processor 118 may also perform at least some steps or operations associated with the tissue identification and/or classification processes independently or in conjunction with one or more other processor(s) 116 and/or CPUs on computing system 104. In some examples, image processor 118 may be or include an FPGA, a digital signal processing (DSP) processor, a graphics processing unit (GPU), or the like. In other examples, processing of the one or more image signals (e.g., raw image data) captured by imaging device 110 may occur entirely on the CPUs of computing system 104.

As mentioned above, in some examples, computing system 104 may include its own GPU or similar processor for executing the image processing (e.g., tensor processing unit (TPU) , neural processing unit (NPU), etc.). In other examples, one or more edge devices, such as edge artificial intelligence (AI) devices, may be integrated with the SOM and FPGA of computing system 104 to facilitate one or more image processing aspects.

In some aspects, computing system 104 may include a system on module 122 that houses various processing components in a compact, integrated package. System on module 122 may include specialized hardware such as specialized CPUs. Additionally or alternatively, system on module 122 may include hardware such as FPGAs GPUs, and/or other processing elements optimized for image analysis and machine learning tasks. System on module 122 may also contain an artificial intelligence (AI) application 124 that implements the machine learning algorithms for detecting and characterizing strictures. AI application 124 may process the image data received from imaging device 110, apply trained neural network models or other types of machine learning algorithms to analyze the images for the presence of strictures, classifies any detected strictures (e.g., as benign or malignant), and generate confidence scores for the classifications. AI application 124 may operate in real-time during medical procedures, providing immediate feedback to clinicians through display device 106.

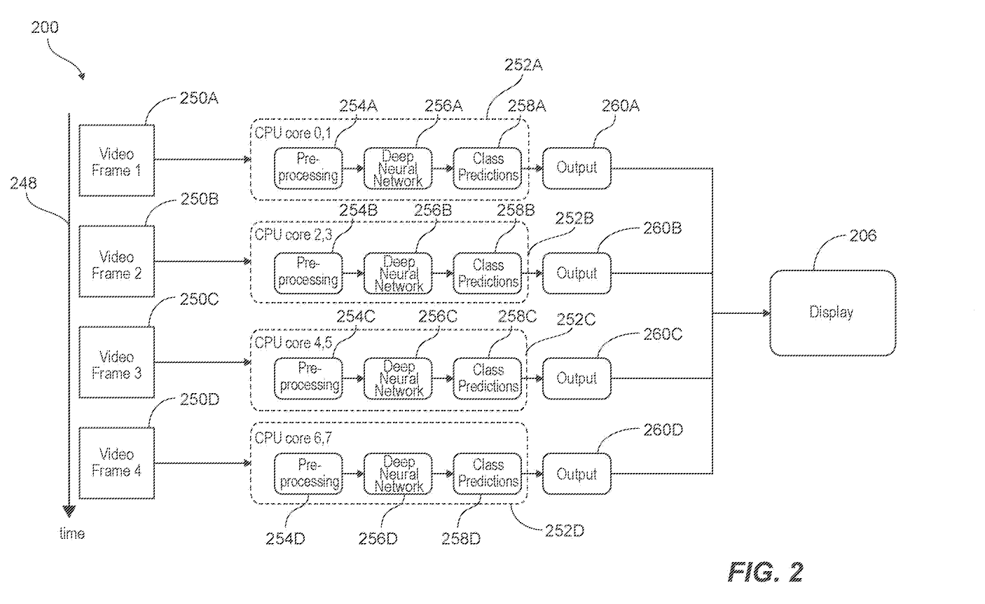

FIG. 2 illustrates an exemplary process 200 for processing and displaying a video frame sequence 248 using AI application 124. In particular, AI application 124 may be configured to analyze video frames in real time, as illustrated in the block diagram of FIG. 2. In some aspects, AI application 124 may process video frame sequence 248 comprising multiple video frames 250A, 250B, 250C, 250D, etc. Although four video frames 250A, 250B, 250C, 250D are shown, fewer video frames or more video frames may be analyzed using AI application 124. Each video frame may be processed by a corresponding processing module 252A, 252B, 252C, and 252D, which may include a central processing unit (CPU) or a core of a CPU. Each processing modules 252A, 252B, 252C, 252D, etc., may be entirely contained within the system on module 122 of computing system 104 (FIG. 1). In other words, processing modules 252A, 252B, 252C, 252D may be housed on-board computing system 104, which may be directly coupled to medical device 102 (e.g., via the umbilicus of medical device 102). Computing system 104 may be located within an endoscopy suite in which a procedure that obtains video frames 250A, 250B, 250C, 250D is performed. To put it another way, processing modules 252A, 252B, 252C, 252D may not be located on a separate device from computing system 104.

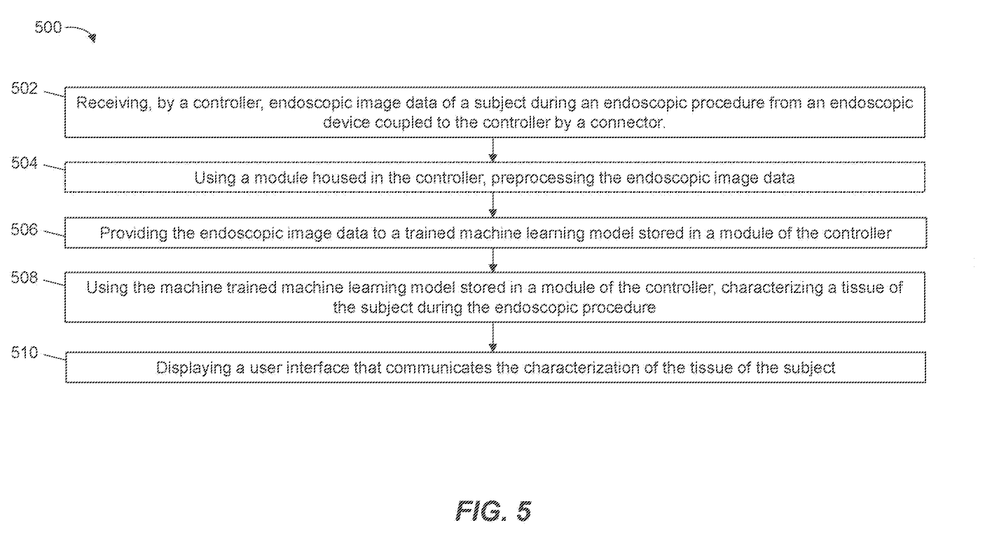

Each of processing modules 252A-D may include a respective preprocessing unit 254A-D, a respective deep neural network 256A-D, and a respective class prediction module 258A-D. Peprocessing units 254A-D may prepare the video frame data for analysis, which may involve tasks such as resizing, normalization, color modification, or feature extraction. The neural networks 256A-D may then process the prepared data. Although the term “neural network” is used in FIG. 2, it will be appreciated that such a term is used for convenience, and other types of machine learning models may be utilized. In some aspects, neural networks 256A-D may utilize convolutional neural network architectures and/or deep neural network architectures optimized for image analysis. Neural networks 256A-D may, in aspects, implement some or all steps of process 500 of FIG. 5, described below. Class prediction modules 258A-D may analyze the output of the neural networks 256A-D to detect and classify strictures or other features of interest within each video frame. Class prediction modules 258A-D may, in aspects, implement some or all steps of process 500 of FIG. 5

Each processing module 252A-D may generate an output 260A-D. Such outputs 260A-D may then be displayed on a display device 206. Outputs 260A-D may include the final processed results, which may include stricture or other lesion classifications, confidence scores, or other relevant information. Outputs 260A-D may be combined to provide a continuous display. In some examples, outputs 260A-D may be displayed consecutively as consecutive video frames. Outputs 260A-D may include video frames 250A-D or may be combined with video frames 250A-250D as video frames 250A-250D are displayed.

In some implementations, processing modules 252A-D may analyze video frames 250A-D in parallel using different CPU cores, which may allow for efficient real-time analysis of multiple video frames simultaneously. The outputs from all processing modules may be combined or synthesized and provided to a display device 206 for presentation to clinicians during the medical procedure and in real time. Display device 206 may have any or all of the characteristics of display device 106 of FIG. 1. In some aspects, display device 206 may be communicatively coupled to the computing system and configured to display processed images and other information.

This real-time video frame analysis capability of process 200 may enable AI application 124 to provide continuous, real-time information about potential strictures or other anatomical features as the medical device 102 is navigated through the patient's body. The parallel processing architecture of process 200 may allow the system to maintain real-time performance even when processing high-resolution or high-frame-rate video streams. Processing modules 252A-D may be configured to handle complex computational tasks efficiently, allowing for sophisticated image analysis techniques to be applied to medical images.

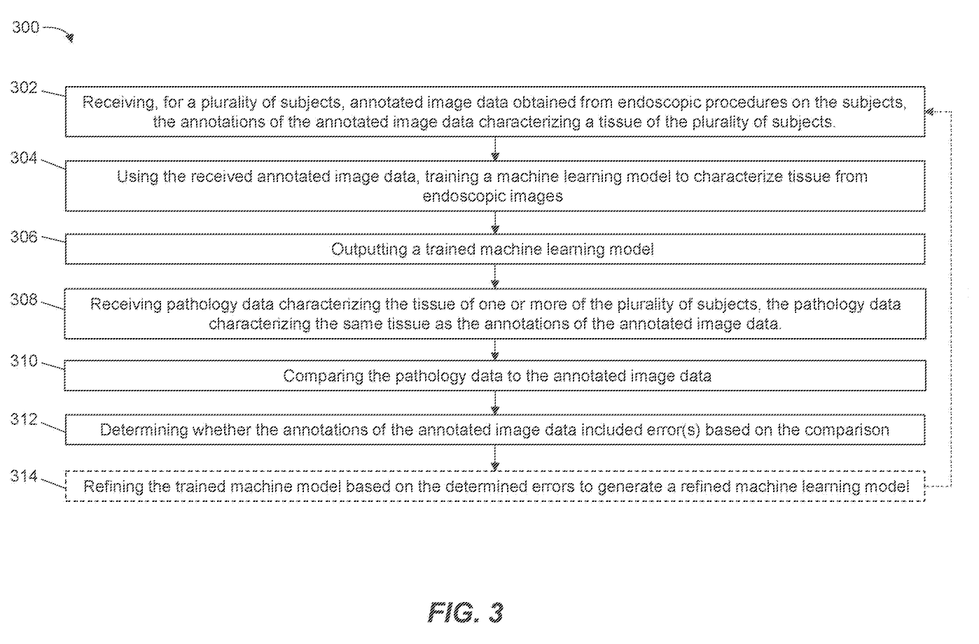

FIG. 3 illustrates an exemplary flowchart of a process 300 for training and refining a machine learning model to characterize tissue from endoscopic images. The process includes several steps that work together to create, validate, and improve the model's performance.

At a first step 302, annotated image data may be received by a computer. For example, step 302 may include receiving, for a plurality of subjects, annotated image data obtained from endoscopic procedures on the subjects, the annotations of the annotated image data characterizing a tissue of the plurality of subjects.

The annotated image data may be obtained from endoscopic procedures performed on one or more subjects. The annotations of the annotated image data may characterize the tissue of the subjects. The annotated image data may include data from various types of endoscopic images, such as those from different parts of the gastrointestinal tract, with corresponding labels indicating tissue types or conditions. The annotated image data may include clearly identifiable strictures, those with subtle or ambiguous stricture presentations, and those without any identifiable strictures at all. By incorporating this diverse spectrum of images, the machine learning models can learn to distinguish between normal anatomical structures and various types of strictures or lesions across different presentations, lighting conditions, and patient anatomies.

In some aspects, the image data received in step 302 can be data that was previously collected during procedures using the trained model, subsequently annotated by clinicians, and then fed back to the model in an iterative learning process. This approach enables continuous improvement of the model using real-world clinical data. For example, the data collected may be used to enhance the model’s accuracy and robustness over time. The data collected may include a variety of images, including images that may or may not contain any identifiable strictures or lesion, ensuring the model learns to distinguish between normal and abnormal anatomies.

In some aspects, the annotations of the annotated image data received in step 302 may be classifications pursuant to the Mendoza classification system, or other classification system(s). The Mendoza classification provides a standardized approach for categorizing biliary strictures based on their endoscopic appearance and characteristics. For example, the Mendoza classification system may be used to identify and assess the severity of malignant and benign lesions, tortuous and dilated vessels, irregular nodulations, raised intraductal lesions, irregular tissue surfaces with or without ulcerations, and/or friability of the tissue. In some aspects, clinicians annotating the image data may use the Mendoza classification to label specific regions or features within the endoscopic images. The annotations may indicate a presence of a stricture or other feature (e.g., a lesion) and a classification of the stricture or other feature. For example, the annotations may indicate whether the stricture or other feature is malignant or benign. The annotations may additionally or alternatively identify a particular type of malignancy or a particular type of benign lesion for a given stricture or other feature. In examples, the annotations may classify a size, severity, risk level, or other characteristic of the stricture or other feature.

Step 304 includes using the received annotated image data to train a machine learning model to characterize tissue from endoscopic images. For example, step 304 may include, using the received annotated image data, training a machine leaning model to characterize tissue from endoscopic images. This step may employ various machine learning techniques, such as deep learning or convolutional neural networks, to process the annotated images and learn patterns associated with different tissue characteristics. The training process may involve feeding the annotated images through multiple layers of the neural network, with each layer extracting increasingly complex features from the images. Initial layers may identify basic elements such as edges and textures, while deeper layers may recognize more complex patterns specific to different types of strictures. Additionally or alternatively, the training process may involve saving images newly captured with or without annotations. The images may be used to train the model, for example, separate from computing system 104. In such aspects, the images may be transmitted to network 140 or stored on computing system 104 and later transferred to a separate system (e.g., a separate computing system or other service side system(s)) to train the model.

The training process typically involves iterative optimization using techniques such as backpropagation and gradient descent to minimize the difference between the model's predictions and the ground truth annotations. During training, the model may be exposed to various data augmentation techniques, such as rotation, scaling, and color adjustments, to improve its robustness and generalization capabilities. The training may also incorporate techniques like transfer learning, where a pre-trained model on a large dataset is fine-tuned using the specific endoscopic image dataset, potentially accelerating the learning process and improving performance when working with limited training data. Cross-validation techniques may be employed to assess the model's performance during training and prevent overfitting to the training data.

In step 306, a trained machine learning model may be an output. For example, step 306 may include outputting a trained machine learning model. This model represents the initial version that has learned to characterize tissue based on the annotated image data. The trained model may be capable of analyzing new, unseen endoscopic images and providing tissue characterizations of the tissue captured in the images. The trained model may be stored in memory 114 of the computing system 104 and/or the system on module 122 (see FIG. 1) for subsequent deployment during medical procedures. In some examples, process 300 may terminate with step 306. In some aspects, step 306 may be omitted or may be performed after step 314.

In some examples, process 300 may continue with a step 308. In some aspects, step 308 may be a first step of a separate method. In examples, steps 302-306 may be performed at a first time, and steps 308-314 may be performed at a later time (by a same computer as steps 302-306 or by a different computer). Step 308 may include receiving pathology data characterizing the tissue of one or more of the plurality of subjects, the pathology data characterizing the same tissue as the annotation of the annotated image data.

In step 308 pathology data that characterizes the tissue of one or more of the subjects may be received by a computer. In aspects, the pathology data characterizes the same tissue as the tissue of the annotated image data received in step 302. In aspects, the pathology data received in step 308 may represent a ground truth. The pathology data may include histopathological findings, biopsy results, surgical specimens, or other diagnostic information. By incorporating pathology data, the machine learning model's accuracy may be validated and/or improved.

At a step 310, the pathology data is compared to the annotated image data. This comparison may reveal discrepancies between the annotations of the annotated image data and the characterization of the pathology data. The comparison process may involve analyzing multiple aspects of the tissue characterization from the annotations of the annotated image data and a pathology report, such as the presence or absence of malignancy, the specific type and/or severity of the stricture, or other clinically relevant features. The comparison may be performed on a case-by-case basis, where each individual subject's annotated image data is directly compared to their corresponding pathology results.

Step 312 involves determining whether the annotations of the annotated image data included error(s) based on the comparison performed in step 310. This step may identify instances where the annotations differ from the pathology results. Additionally, step 312 may categorize errors based on their clinical significance.

Finally, at step 314, the process refines the trained machine model based on the determined errors to generate a refined machine learning model. For example, this error determination process may involve calculating various metrics such as false positive rates, false negative rates, sensitivity, specificity, and overall accuracy across different types of strictures. This refinement may involve adjusting the model's parameters, updating the training data, or modifying the model architecture to improve its accuracy in tissue characterization. The refinement process may include techniques such as adjusting learning rates, implementing regularization methods to prevent overfitting, or incorporating additional layers or nodes within the neural network architecture. In some implementations, the refinement may involve retraining the model with corrected annotations based on the pathology data.

In some aspects, the refinement process may also include data augmentation techniques to enhance the model's ability to generalize across different patient populations, imaging conditions, or stricture types. Additionally or alternatively, the refinement may involve implementing ensemble methods that combine multiple models to improve overall prediction accuracy. The refined machine learning model resulting from this step may demonstrate improved sensitivity and specificity in detecting and characterizing strictures, helping to produce more accurate characterizations, which may be used by operators to guide clinical decisions.

This cyclical methodology of training, validation, and refinement may create a continuous improvement loop that may enhance the model's capabilities for tissue characterization from endoscopic images in real time. By repeatedly exposing the model to new pathology data and comparing its predictions against confirmed diagnoses, the system progressively reduces classification errors and improves diagnostic accuracy.

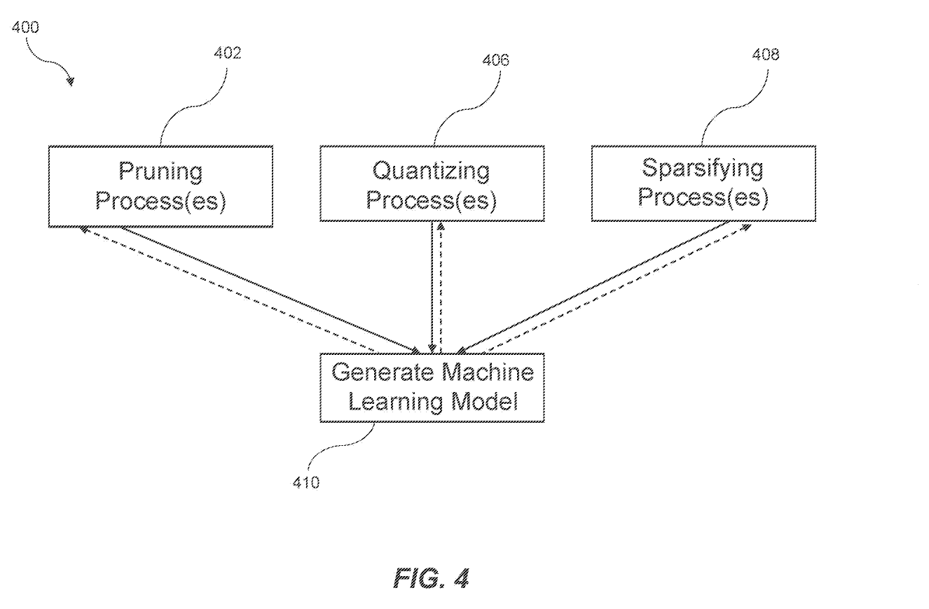

FIG. 4 depicts a block diagram of a model generation methodology 400. Methodology 400 includes multiple processes that may be used to generate a machine learning model 410. For example, model generation methodology 400 may incorporate pruning process(es) 402, quantizing process(es) 406, and/or sparsifying process(es) 408. These process(es) may be utilized to generate model 410. Such processes may be used in any combination, for example, to enhance the efficiency of machine learning model 410. In aspects, methodology 400 may be performed concurrently with process 300 (e.g., during step 304 or step 306). Alternatively, methodology 400 may be implemented after process 300. In some aspects, machine learning model 410 may be an output of process 300 or may otherwise embody the training of process 300.

Pruning process(es) 402 may be used to reduce the processing power required to run machine learning model 410 by removing unnecessary or redundant components. This process may help optimize the model's performance and efficiency. Pruning process(es) 402 may involve systematically eliminating connections, neurons, or filters within the neural network that contribute minimally to the model's overall performance. By identifying and removing these less important elements, the pruning process(es) 402 can significantly reduce the computational requirements and memory footprint of machine learning model 410 without substantially degrading its accuracy. Additionally, the pruning process(es) 402 may implement iterative pruning strategies, where the model is pruned gradually over multiple iterations, with retraining between pruning steps to recover any lost accuracy.

Quantizing process(es) 406 may be employed to reduce the precision of the model's parameters, potentially decreasing the computational requirements and memory footprint of machine learning model 410 without significantly impacting its accuracy. Quantization involves converting high-precision floating-point values (such as 32-bit or 64-bit) to lower-precision representations (such as 16-bit, 8-bit, or even binary values). This process can substantially reduce the memory requirements and computational complexity of the machine learning model 410, making it more suitable for deployment on resource-constrained hardware typically found in medical devices. The quantizing process(es) 406 may implement various quantization techniques, such as post-training quantization, where a pre-trained model is quantized after training is complete, or quantization-aware training, where the model is trained with quantization effects in mind. Different layers of the neural network may be quantized to different precision levels based on their sensitivity to quantization errors, with more critical layers potentially retaining higher precision. The quantizing process(es) 406 may also incorporate techniques or processes to minimize the accuracy loss during quantization, such as calibration methods that adjust the quantization parameters based on representative data samples (e.g., training data). Additionally or alternatively, post-training accuracy-aware quantization processes may be implemented. In some aspects, various parts of the architecture associated with quantizing process(es) 406 may be quantized by different amounts.

Sparsifying process(es) 408 may introduce sparsity into machine learning model 410, potentially reducing the number of non-zero parameters and further optimizing the model's structure. Sparsification may include strategically converting a portion of the model's parameters to zero values while preserving the model's overall performance capabilities. This process can significantly reduce the computational requirements and memory footprint of machine learning model 410, making it more efficient for deployment on resource-constrained hardware typically found in medical devices. Sparsifying process(es) 408 may implement various sparsification techniques, such as magnitude-based pruning, where parameters below a certain threshold are set to zero, or structured sparsity, where entire groups of parameters are sparsified together to maintain computational efficiency.

Additionally or alternatively, the sparsifying process(es) 408 may employ regularization techniques during training that encourage sparsity. Sparsifying process(es) 408 may be applied selectively to different parts of the neural network based on their sensitivity to sparsification, with less critical connections being more aggressively sparsified. By carefully introducing sparsity, the system can achieve substantial reductions in model size and computational requirements while maintaining high accuracy in stricture detection and classification tasks.

These processes - pruning process(es) 402, quantizing process(es) 406, and sparsifying process(es) 408 - may work individually or in concert to refine and optimize the machine learning model 410. The methodology 400 may utilize these techniques to generate a machine learning model that balances performance, efficiency, and accuracy for the specific task of analyzing endoscopic images and characterizing tissue. These optimization processes may reduce the computational complexity and memory requirements of machine learning model 410, for example, without significantly compromising its accuracy, making it suitable for deployment on the resource-constrained computing systems typically found in medical devices. Such processes may be performed on a CPU of computing system 104. In aspects, computing system 104 may be directly coupled to the scope (e.g., via communication interface 120).

In some aspects, the process of generating and refining the machine learning model 410 may be iterative. This iterative nature is illustrated in FIG. 4 by the broken lines extending between the machine learning model 410 and each of the pruning process(es) 402, quantizing process(es) 406, and sparsifying process(es) 408. These broken lines may indicate that the machine learning model 410 may undergo multiple rounds of pruning, quantizing, and sparsifying processes as part of an ongoing optimization process. The iterative approach may allow for continuous refinement of the machine learning model 410. After an initial round of pruning, quantizing, and sparsifying, the resulting model may be evaluated for performance and accuracy. Based on this evaluation, the model may be fed back through one or more of the pruning process(es) 402, quantizing process(es) 406, or sparsifying process(es) 408 for further optimization. This process may be repeated multiple times, with each iteration potentially improving the model's efficiency, reducing its computational requirements, or enhancing its performance. Furthermore, in some aspects, this iterative approach may also allow the system to adapt the machine learning model 410 to new data or changing requirements over time. As new endoscopic images become available or as the understanding of stricture characteristics evolves, the model may be iteratively refined using these techniques to maintain its relevance and accuracy.

FIG. 5 illustrates an exemplary flowchart depicting a process 500 for processing endoscopic image data. Process 500 may be performed by the machine learning model trained according to process 300, using the techniques of FIG. 4. The trained machine learning model used in process 500 may be implemented as a convolutional neural network (CNN), a vision transformer (ViT), a deep neural network, or another suitable architecture optimized for image analysis. Process 500 may be configured to output various types of characterizations, such as binary classifications (e.g., benign versus malignant), multi-class classifications (e.g., different types of strictures according to the Mendoza classification system), and/or severity scores of the tissue.

For example, at a first step 502, a controller may receive endoscopic image data of a subject obtained during an endoscopic procedure from an endoscopic device (e.g., medical device 102 of FIG. 1) coupled to the controller (e.g., computing system 104 of FIG. 1) by a connector. In some aspects, step 502 may be performed by another type of device (e.g., an offboard device or a cloud-based system). In aspects, the endoscopic procedure may be an ERCP procedure. This step may involve the real-time acquisition of image data as the endoscopic procedure is being performed. The image data received may be individual frames taken from video data over the course of the procedure (as described with respect to FIG. 3).

In a step 504, endoscopic image data may be pre-processed, as described with reference to FIG. 3. Step 504 may be optional (along with any other step of any other method disclosed herein). For example, step 504 may include preprocessing the endoscopic image data by using a module housed in the controller. In aspects, using a module housed in the controller may be performed by system on module 122 of computing system 104. This preprocessing step may include various operations to prepare the image data for analysis, such as noise reduction, contrast enhancement, or feature extraction. The preprocessing may also involve image normalization to standardize brightness and color values across different endoscopic images, image resizing to ensure consistent dimensions for input to the machine learning model, segmentation to isolate regions of interest that may contain strictures, edge detection to highlight boundaries between different tissue types, and color space transformations to emphasize specific visual characteristics of potential strictures. These preprocessing operations may improve the quality and consistency of the input data, which may impact the accuracy and reliability of the subsequent tissue characterization performed by the machine learning model in a step 508, described below.

A step 506 incudes providing the preprocessed endoscopic image data to a trained machine learning model stored in a module of the controller (e.g., system on module 122 of computing system 104). This step may transfer the prepared image data to the machine learning model for analysis. The transfer of preprocessed data to the trained machine learning model may occur through internal memory transfers within the controller, ensuring minimal latency for real-time analysis during the endoscopic procedure. The trained machine learning model may be stored in a dedicated module of the controller, such as the system module 122 described with reference to FIG. 1, which may include specialized hardware optimized for machine learning tasks. The model may be implemented as a convolutional neural network (CNN) or other learning architecture specifically trained to recognize and classify strictures, as described above. The preprocessed endoscopic image data may be formatted according to the specific input requirements of the trained model, including appropriate dimensionality, color space, and normalization parameters.

At step 508, the trained machine learning model trained in process 300 and stored in a module of the controller (e.g., system on module 122 of computing system 104) is used to characterize a tissue of the subject during the endoscopic procedure. The machine learning model may analyze the preprocessed image data to identify and classify any strictures, lesions, or other relevant tissue characteristics. The characterization process may include detecting the presence of strictures, determining their location within the biliary or pancreatic ductal systems, classifying the strictures (e.g., according to a Mendoza classification system), and assessing a likelihood of malignancy for observed tissue features.

During step 508, the trained machine learning model may generate confidence scores for different classifications, such as benign versus malignant, which can be presented to the clinician as part of the analysis results in a step 510. Step 508 may be a real-time tissue characterization that occurs while the endoscopic procedure is ongoing. In aspects, the real-time nature of step 508 may facilitate immediate clinical decision-making. The characterization may be continuously updated as new images are captured during the procedure.

In some embodiments, the output of the trained machine learning model, such as a confidence score or classification probability for detected tissue features, may be aggregated over a series of consecutive endoscopic image frames or video frames captured during the procedure. This aggregation may involve averaging the confidence scores across a sliding temporal window of N frames to produce a smoothed or stabilized output. The aggregated confidence score may then be compared to one or more predefined thresholds to determine a final tissue characterization or to trigger alerts within the user interface.

For example, by averaging model outputs over multiple frames, transient fluctuations or noise in individual frame predictions may be mitigated, resulting in enhanced reliability and reduced false positive or false negative rates. The size of the temporal window N and the threshold values may be configurable based on clinical requirements or user preferences. This temporal smoothing technique facilitates more robust real-time feedback to the operator, improving diagnostic confidence during ongoing endoscopic procedures.

In some embodiments, the user interface may be configured to display both the real-time confidence score generated by the machine learning model for the current endoscopic frame as well as an averaged confidence score computed over a series of N consecutive frames or over a time interval T during the procedure. The averaged confidence score may be presented alongside the real-time score to provide clinicians with both instantaneous and stabilized assessments of the tissue characterization.

Displaying the averaged confidence score may involve overlaying numerical values, color-coded indicators, or graphical elements such as progress bars or heatmaps on the endoscopic image data to visually differentiate between dynamic frame-to-frame predictions and the smoothed, aggregated output. For example, the averaged score may use a distinct color or a secondary display area within the interface to highlight sustained patterns indicating malignancy or benign features.

The parameter N, representing the number of frames, or T, representing the time duration over which the averaging occurs, may be adjustable based on clinical workflow or operator preference, allowing for real-time control over the trade-off between responsiveness and stability of the displayed tissue characterization. By incorporating both the real-time and averaged scores in the user interface, the system can improve diagnostic confidence and support more informed clinical decision-making during ongoing endoscopic procedures.

In some embodiments, the user interface may dynamically change color or display visual indicators when either the real-time confidence score, the averaged confidence score, or both exceed predefined threshold values. For example, the overlay outlining the tissue region of interest or the background color of the confidence score display may transition to a distinct color—such as red to indicate high likelihood of malignancy or green to indicate benign status—when the threshold is met or exceeded. Additionally, the UI may present icons, flashing signals, or textual alerts to draw the clinician’s attention to significant findings.

These threshold-triggered visual cues may be configured independently for real-time and averaged scores, enabling nuanced feedback that distinguishes transient detections from sustained results. The thresholds and corresponding UI response behaviors may be customizable to suit different procedural contexts or user preferences, thereby providing intuitive and immediate notification of critical tissue characterizations during endoscopic procedures.

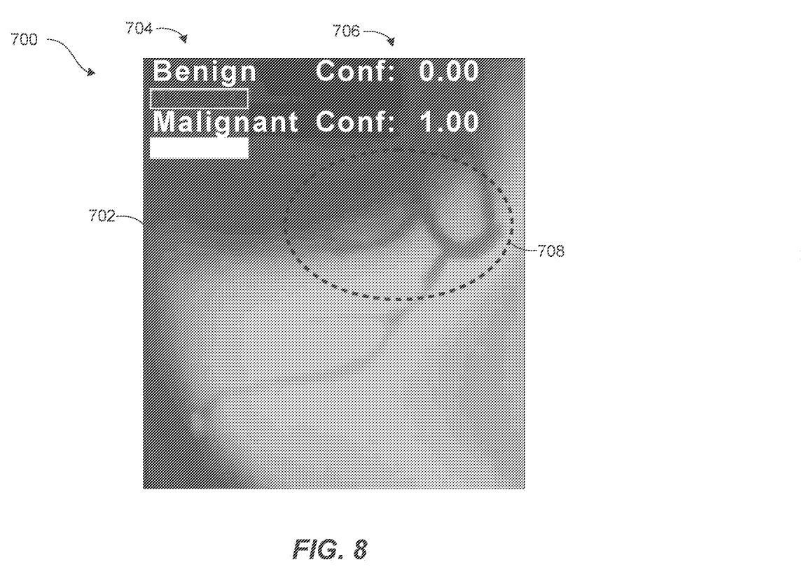

Step 510 includes displaying a user interface that communicates the characterization of the tissue from step 508 of the subject. Step 510 may present the results of the trained machine learning model’s analysis from step 508 to the clinician in a clear and interpretable format. In step 510, the model may also identify specific visual features that contributed to its classification decision, potentially highlighting areas of interest within the endoscopic image. In aspects the user interface may include visual or graphic overlays on displayed endoscopic images (e.g., live images and/or still images) or numerical confidence scores for tissue classifications. The user interface may display confidence scores for one or more classifications, such as benign versus malignant, with numerical values ranging from 0.00 to 1.00 as shown in FIG. 8. In some examples, the confidence scores may indicate the likelihood of observed tissue features as being benign or malignant.

The interface may also include color-coded indicators where, for example, higher confidence scores for malignancy might be displayed in red, while higher confidence scores for benign conditions might be displayed in green, thus providing dynamic feedback as the endoscope navigates through the patient's anatomy. In some aspects, the confidence scores may be compared against a threshold. For example, if a confidence score meets or exceeds the threshold, the condition may be used as a computer-aided diagnosis (e.g., a CADx).

In some implementations, the user interface may highlight regions of interest within the endoscopic image where strictures are detected, potentially outlining these areas or applying semi-transparent colored overlays to indicate the specific locations of detected abnormalities. The interface may also display additional contextual information, such as classifications according to established classification systems like the Mendoza classification, estimated dimensions of the stricture, or suggested next steps for diagnosis or treatment based on the machine learning model's analysis. This comprehensive visualization may helps to enable clinicians to quickly interpret the machine learning model's findings during the procedure, potentially improving decision-making efficiency and accuracy while maintaining the clinician's autonomy in determining the final diagnosis and treatment approach. The interface may update continuously, for example, as the scope moves and displays different images.

In some aspects, the user interface may include a heatmap of malignancies. For example, the image may be broken into tiles. A mask may be applied to one or more tiles, and an output prediction may be generated for the resulting image (e.g., the image with the one or more tiles that were masked). This process may be repeated until each tile has been masked. The output predictions for each may be compared to one another and/or compared to the output prediction of the overall image (e.g., without any tiles masked) to determine which tile contains malignant or benign features.

By way of example, a first image without any tiles masked may have a first output prediction for malignancy. When that image is divided into tiles and at least one tile is masked (e.g., referred to as a second image), the second image may have a second output prediction for malignancy. If the output prediction for malignancy of the first image is higher than the output prediction for malignancy in the second image, it may be determined that the at least one tile that was masked in the second image contains features that are malignant. The user interface may then apply an overlay over that at least one tile. For example, the overlay may be any color or shape to indicate the presence of malignant features within that tile. Likewise, if the output prediction for malignancy of the first image is lower than the output prediction for malignancy in the second image, it may be determined that the at least one tile that was masked in the second image does not contain features that are malignant. The user interface may then apply an overlay of a second color or shape to indicate the absences of malignant features. If the output prediction for malignancy is the same between the first and second images, a third color or shape may be overlayed or no overlay be applied. In other aspects when the output prediction for malignancy is the same between the first and second images, the output prediction may be compared to a threshold an appropriate overlay may be applied. For example, if the output prediction is greater than or equal to 0.5 in the first and second image, the first color, indicating malignancy, may be applied as an overlay. This process may be repeated until all or a portion of the image includes the overlay.

Process 500 illustrates how endoscopic image data may be processed and analyzed in real-time using machine learning techniques, enabling immediate feedback and decision support during ongoing endoscopic procedures. This real-time processing occurs while the physician is actively performing the procedure, allowing for instantaneous detection and characterization of strictures as they come into view. Computing system 104, as shown in FIG. 1, performs this real-time analysis using the system on module 122 which houses AI application 124. As described above, the AI application 124 implements sophisticated machine learning algorithms specifically optimized for detecting and characterizing strictures according to established classification systems such as the Mendoza classification. The system on module 122 contains specialized hardware components that enable high-speed processing of endoscopic images without introducing latency that would impede clinical workflow. This integrated approach may significantly enhance the ability of clinicians to detect and characterize strictures or other tissue abnormalities accurately and efficiently, potentially improving diagnostic consistency across different endoscopists and enabling more informed treatment decisions during the procedure itself rather than requiring follow-up analysis.

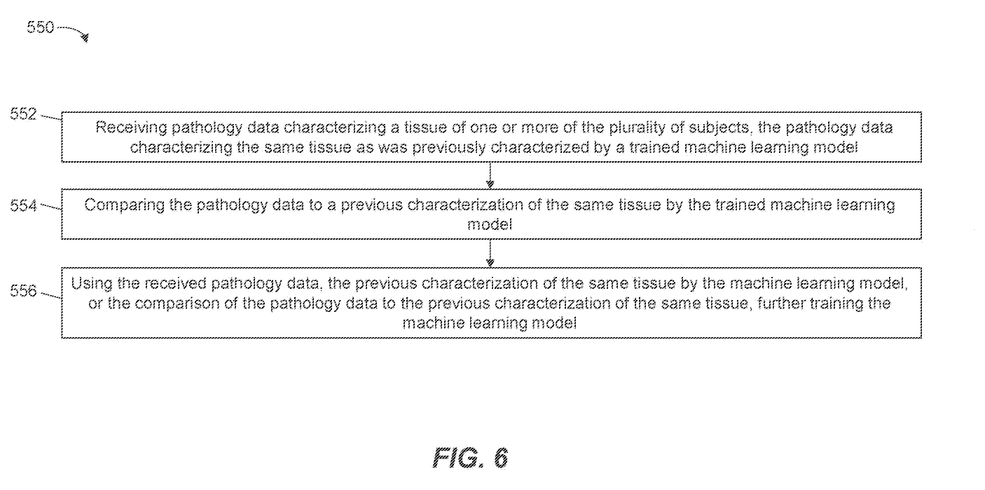

FIG. 6 illustrates an exemplary flowchart of a process 550 for processing pathology data to refine and validate the tissue characterization capabilities of the trained machine learning model utilized in process 500.

At a first step 552, process 550 may include receiving pathology data that characterizes a tissue of one or more of the plurality of subjects. This pathology data may correspond to the same tissue that was previously characterized by a trained machine learning model in process 500. The pathology data may include histological findings, biopsy results, or other definitive diagnostic information obtained through standard medical procedures.

Following the receipt of pathology data, a second step 554 may include comparing the received pathology data to the previous characterization of the same tissue that was made by the trained machine learning model (e.g., via process 500). This comparison step may help identify any discrepancies between the model's predictions and the ground truth provided by the pathology data. The comparison process may involve detailed analysis of multiple aspects of tissue characterization, such as the presence or absence of malignancy, the specific type of stricture, or the severity of the stricture. Statistical methods may be employed to quantify the agreement between the model's predictions and the pathology results, potentially including metrics such as sensitivity, specificity, positive predictive value, and negative predictive value. The comparison may be performed on both individual cases and across a dataset having multiple patients to identify patterns of agreement or disagreement. This comprehensive comparison may highlight specific areas where the model has weaknesses (e.g., misclassifying types of strictures or strictures in particular anatomical locations). Additionally, the comparison process may categorize discrepancies based on their clinical significance, prioritizing refinements that would have the greatest impact on patient outcomes and treatment decisions.

At a step 556, the process 550 includes using the received pathology data, the previous characterization of the same tissue made by the machine learning model, or the comparison of the pathology data to the previous characterization of the same tissue to further train the machine learning model. Step 556 may involve adjusting the model's parameters, updating its training data, or modifying its architecture to improve its accuracy in tissue characterization based on the insights gained from the pathology data. The further training may include fine-tuning the weights and biases of the neural network to better align with the pathology results, particularly for cases where discrepancies were identified during the comparison step. In some implementations, this further training may involve transfer learning techniques, where the existing model serves as a starting point but is refined using the pathology data as a new source of ground truth. The training process may employ techniques such as gradient descent optimization, backpropagation, or regularization methods to systematically improve the model's performance. Additionally, the further training may incorporate ensemble methods, where multiple models or model variations are combined to enhance overall prediction accuracy. The training may also involve data augmentation techniques to expand the effective training dataset based on the pathology findings. This iterative refinement process enables the machine learning model to continuously improve its ability to characterize tissue from endoscopic images, potentially leading to more accurate and consistent diagnoses across different endoscopists and clinical settings. The refined model resulting from step 556 may demonstrate improved sensitivity and specificity in detecting and classifying strictures according to established classification systems such as the Mendoza classification.

By incorporating definitive pathology results, this process may enable continuous improvement of the model's performance, potentially leading to more accurate and reliable tissue characterization during endoscopic procedures. Process 550 may be repeated to create a feedback loop where the model's predictions are systematically compared against pathology findings, allowing for targeted refinements in areas where discrepancies are identified. The integration of pathology data provides an objective reference standard against which the model's performance can be measured. Additionally, this approach may enable the model to adapt to new or evolving patterns of stricture presentation over time, as continued exposure to diverse pathology data may enhance the model's ability to recognize rare or atypical cases. The systematic comparison between machine learning predictions and pathology results may also generate valuable insights into the specific visual features or patterns that most reliably indicate malignancy.

This iterative refinement process enables the model to continuously learn from new pathology data and clinical feedback, making it increasingly effective at characterizing tissue from endoscopic images over time. Furthermore, in some aspects, statistical analysis may be performed to potentially identify systematic biases in the model's predictions, such as consistently misclassifying certain types of strictures or having difficulty with specific anatomical locations. The error analysis may also consider the confidence levels associated with each prediction, potentially revealing cases where the model was highly confident but incorrect,. Such an analysis may also be used to correct the model’s predictions, if necessary.

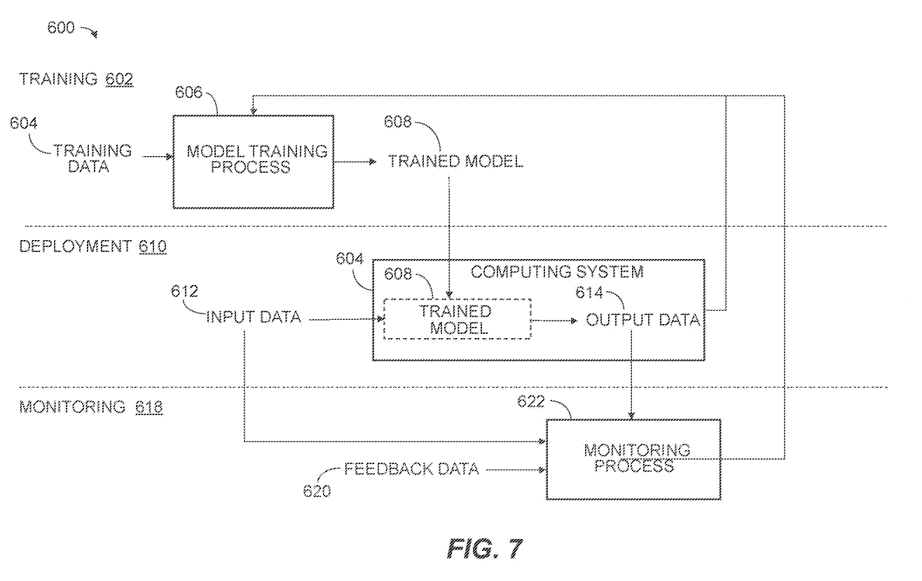

FIG. 7 is an exemplary block diagram showing an example process 600 for training, using, and monitoring a machine learning model to identify and/or classify or characterize strictures. In some aspects, a system or device other than computing system 104 may be used to generate and/or train the machine learning model. For example, one or more of server-side system(s) 130 may include instructions for generating and/or training the machine learning model. A resulting trained machine learning model is then provided to computing system 104, in particular to system on module 122, for storage and use with AI application 124. In other aspects, the computing system 104 itself generates, trains, stores, and uses the machine learning model.

As depicted in FIG. 7, in some examples, process 600 may include a training phase 602, a deployment phase 610, and a monitoring phase 618. Training phase 602 may include any of the aspects of processes 300 or 550, using any of the aspects of methodology 400. Process 300 and methodology 400 may implement any of the aspects of training phase 602 described herein. Deployment phase 610 may include any of the aspects of process 500, and process 500 may include any of the aspects of deployment phase 610. Monitoring phase 618 may include any of the aspects of process 550, and process 550 may include any of the aspects of monitoring phase 618. Monitoring phase 618 may also include aspects of process 300, and process 300 may include aspects of monitoring phase 618. In training phase 602, step 606 of process 600 may include receiving and processing certain training data 604 to generate (e.g., build) a trained machine learning model 608 for identifying and/or classifying or characterizing strictures.

Training data 604 may include endoscopic images, video frames, and corresponding annotations that characterize tissue types, stricture classifications, and pathology results from one or more endoscopic procedures. For example, the training data may be collected from multiple sources, including historical patient records, clinical trials, public medical image databases, and ongoing procedures performed using the system.

The data may include images showing various types of strictures identified according to established classification systems such as the Mendoza classification, along with expert annotations indicating benign or malignant status. The training data (e.g., images) may include those with clearly identifiable strictures, those with subtle or ambiguous stricture presentations, and those without any identifiable strictures at all. By incorporating this diverse spectrum of images, the machine learning models can learn to distinguish between normal anatomical structures and various types of strictures across different presentations, lighting conditions, and patient anatomies. The training data may also include negative examples showing normal anatomy without strictures to help the model distinguish between normal and abnormal tissue. Additionally, the training data may incorporate demographic information, patient history, and other contextual factors that might influence stricture characterization. To ensure robustness, the training data typically includes images captured under various lighting conditions, from different angles, and using different endoscopic equipment. In aspects, training data 604 may be generated, received, or otherwise obtained from internal and/or external resources.

Generally, a model includes a set of variables, e.g., nodes, neurons, filters, etc., that are tuned, e.g., weighted or biased, to different values via the application of training data 604. In some examples, the training process performed at step 606 employs supervised, unsupervised, semi-supervised, and/or reinforcement learning processes to train the model (e.g., to result in trained machine learning model 608). In some aspects, a portion of training data 604 is withheld during training and/or used to validate trained machine learning model 608.

When supervised learning processes are employed, annotations, labels, or scores facilitate the learning process by providing a ground truth. For example, the annotations, labels, or scores indicate whether a particular stricture is present or absent in image, and/or one or more characteristics of that stricture. Training proceeds by feeding a training image (e.g., a sample) into the model, the model having variables set at initialized values, e.g., at random, based on Gaussian noise, a pre-trained model, or the like. The model outputs a score and/or a probability of the presence and/or type of stricture present (e.g., benign or malignant). In some aspects, the output may include a Mendoza score (and/or the probability of a specific score), potentially indicating the severity of the stricture. The output is compared with the corresponding annotation, label, or score (e.g., the ground truth) to determine an error, which is then back-propagated through the model to adjust the values of the variables. This process is repeated for a plurality of samples at least until a determined loss or error is below a predefined threshold. In some examples, some of training data 604 is withheld and used to further validate or test trained machine learning model 410 (FIG. 4).

For unsupervised learning processes, training data 604 does not include pre-assigned annotations, labels, or scores to aid the learning process. Rather, unsupervised learning processes include clustering, classification, or the like to identify naturally occurring patterns in training data 604. Supervised or unsupervised K-means clustering or K-Nearest Neighbors can also be used. Combinations of K-Nearest Neighbors and an unsupervised cluster technique can also be used. For semi-supervised learning, a combination of training data 604 with pre-assigned annotations, labels, or scores and training data 604 without pre-assigned annotations, labels, or scores are used to train the model.

When reinforcement learning is employed, an agent (e.g., an algorithm) is trained to make a decision regarding the identification and/or classification of a structure from training data 604 through trial and error. For example, upon making a decision, the agent then receives feedback (e.g., a positive reward if the predicted output was agreed with by the operator), adjust its next decision to maximize the reward, and repeat until a loss function is optimized.

Once trained, trained machine learning model 608 is stored (e.g., in memory 114 of FIG. 1) and subsequently executed by computing system 104 (FIG. 1) during deployment phase 610. For example, during deployment phase 610, trained machine learning model 608 receives input data 612. Trained machine learning model 608 outputs output data 614. Furthermore, the stored machine learning model 608 may be used for subsequent training purposes.

During monitoring phase 618, feedback data 620 is collected. Feedback data 620 includes confirmation of the presence of a stricture and/or confirmation of the classification of the stricture identified. Confirmation may be further enforced using the model, pathological data, and/or other historical data from the patient. During monitoring process 622, feedback data 620 is analyzed along with input data 612 to determine an accuracy of output data 614. In some examples, based on the analysis, process 600 returns to training phase 602, where at step 606 the values of one or more variables of trained machine learning model 608 are adjusted to re-train the model. For example, new training data may be generated based on input data 612 and feedback data 620 (e.g., feedback data 620 may be utilized as an annotation, label, or score for input data 612), and fed into trained machine learning model 608 to cause the adjusting of the variables.

The process 600 described above is provided merely as an example, and can include additional, fewer, different, or differently arranged aspects than depicted in FIG. 7.

FIG. 8 illustrates an exemplary user interface 700. User interface 700 may include an image 702. Additional information may be overlayed over image 702. For example, a first graphic, or visual, overlay 704. First graphic overlay 704 may include a classification category, such as "Benign" and "Malignant," although any other classification category may be utilized.

User interface 700 may further include a second graphic, or visual, overlay 706, which may include confidence scores associated with the classification categories of first graphic overlay 704. For example, as shown in FIG. 8, first graphic overlay 704 and second graphic overlay 706 are displayed prominently at the top of image 702 of user interface 700. By way of example, the “benign” category of first graphic overlay 704 shows a score of 0.00 for the second graphic overlay 706. The “malignant” category of first graphic overlay 704 shows a score of 1.00 for the second graphic overlay 706. In aspects, the confidence scores may range from 0.00 to 1.00, where 1 represents 100 % confidence. In this example, the system displays high confidence (1.00) for the "Malignant" classification and low confidence (0.00) for the "Benign" classification, suggesting that the analyzed image likely contains features strongly indicative of a malignant stricture. User interface 700 may thus assist clinicians in making rapid, informed decisions during endoscopic procedures by providing immediate feedback on the likelihood of malignancy for observed strictures or tissue features.

In aspects, user interface 700 may further include a third graphic, or visual, overlay 708, which may include a broken or solid outline that surrounds the tissue being classified. Third graphic overlay 708 may assist clinicians in identifying the target tissue. The tissue shown within graphic overlay 708 may correspond to the tissue that is being classified, as described above. In some aspects, third graphic overlay may be colored. For example, the outline may be red to indicate the tissue within the outline is likely malignant (e.g., a “malignant” confidence score greater than or equal to 0.75 or any other set threshold), the outline may be green to indicate the tissue within the outline is likely benign (e.g., a “benign” confidence score greater than or equal to 0.75 or any other set threshold), and/or the outline may be yellow to indicate the tissue could be malignant or benign (e.g., at least one of the “malignant confidence score or the “benign” confidence score is less than 0.75 or any other set threshold).

Additionally or alternatively, user interface 700 may include color overlays to similarly assist clinicians in identifying the target tissue. Such overlays may include any colors that may assist a clinician with quickly identifying the tissue being classified. Similarly, the tissue that includes a color gradient may correspond to the tissue that is being classified, as described above. For example, malignant tissues features may be colored red, whereas benign tissue features may be colored yellow or green, or vice versa. The colored overlays may be any desired color and, in some aspects, may be transparent or transparent, such that the clinician is able to still see the tissue under the overlay.

Image 702 may be a live endoscopic view that is being analyzed by the system in real time. By showing both the live endoscopic image and the corresponding categories and confidence scores simultaneously, user interface 700 may allow clinicians to quickly interpret the model's assessment of any observed strictures. User interface 700 may be continuously updated in real-time, as discussed above.

One or more graphic overlays 704, 706, 708 may be turned on or off manually or automatically. For example, graphic overlays 704, 706, 708 may be manually turned on by the clinician before or during a procedure, and/or graphic overlays 704, 706, 708 may be automatically turned on upon the detection of a stricture during a procedure. Similarly, graphic overlays 704, 706, 708 may be manually turned off by the clinician and/or may be turned off automatically (e.g., after a period of time in which a stricture is not identified). The machine learning processes described above may continue to run in the background when graphic overlays are turned on and/or off. In other aspects, the machine learning processes described above may only be turned on when graphic overlays 704, 706, 708 are turned on, or vice versa.

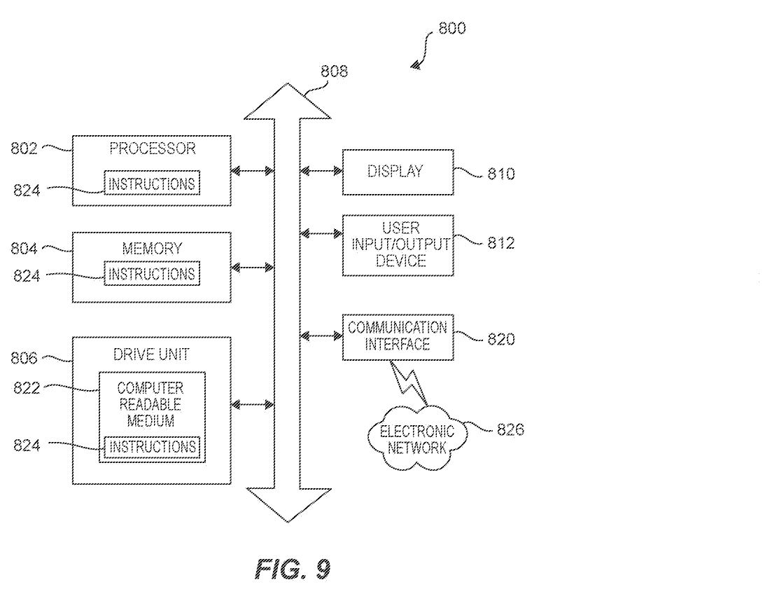

FIG. 9 is a simplified functional block diagram of computer 800 that may be configured as a device for executing processes, steps, or operations depicted in, or described with respect to FIGS. 2-7 and, according to exemplary aspects described herein. For example, computer 800 may be configured as one or more of medical device 102, computing system 104, display device(s) 106, server-side system(s) 130, and/or another device or component according to exemplary aspects described herein. In various aspects, any of the systems herein may be or include computer 800 including, e.g., a data communication interface 820 for packet data communication. Computer 800 may communicate with one or more other computers, for example, using an electronic network 826 (e.g., via data communication interface 820). Electronic network 826 may include a wired or wireless network, for example, similar to network 140 depicted in FIG. 1.

Computer 800 also may include a central processing unit (“CPU”), in the form of one or more processors 802, for executing program instructions 824. In some examples, processors 802 may be or include one or more field-programmable gate arrays (FPGAs). Program instructions 824 may include at least instructions for performing usage monitoring (e.g., if computer 800 is computing system 104).

Computer 800 may include an internal communication bus 808. Computer 800 may also include a drive unit 806 (such as read-only memory (ROM), hard disk drive (HDD), solid-state disk drive (SDD), etc.) that may store data on a computer readable medium 822 (e.g., a non-transitory computer readable medium), although computer 800 may receive programming and data via network communications. Computer 800 may also have a memory 804 (such as random-access memory (RAM)) storing instructions 824 for executing techniques presented herein. It is noted, however, that in some aspects, instructions 824 may be stored temporarily or permanently within other modules of computer 800 (e.g., processor 802 and/or computer readable medium 822). Instructions 824 may be stored for subsequent training of the machine learning model.

Computer 800 also may include user input and output devices 812 and/or a display 810 to connect with input and/or output devices such as keyboards, mice, touchscreens, monitors, displays, etc. The various system functions may be implemented in a distributed fashion on a number of similar platforms, to distribute the processing load. Alternatively, the systems may be implemented by appropriate programming of one computer hardware platform.

Program aspects of the technology may be thought of as “products” or “articles of manufacture” typically in the form of executable code and/or associated data that is carried on or embodied in a type of machine-readable medium. “Storage” type media include any or all of the tangible memory of the computers, processors or the like, or associated modules thereof, such as various semiconductor memories, tape drives, disk drives and the like, which may provide non-transitory storage at any time for the software programming. All or portions of the software may, at times, be communicated through the Internet or various other telecommunication networks. Such communications, e.g., may enable loading of the software from one computer or processor into another. Thus, another type of media that may bear the software elements includes optical, electrical, and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

While principles of the disclosure are described herein with reference to illustrative examples for particular applications, it should be understood that the disclosure is not limited thereto. Those having ordinary skill in the art and access to the teachings provided herein will recognize additional modifications, applications, and substitution of equivalents all fall within the scope of the examples described herein. Accordingly, the invention is not to be considered as limited by the foregoing description.

1. A method for processing endoscopic image data, comprising:
receiving, by a controller, endoscopic image data of a subject during an endoscopic procedure from an endoscopic device coupled to the controller by a connector;
preprocessing, by a module housed in the controller, the endoscopic image data;
providing the preprocessed endoscopic image data to a trained machine learning model stored on the controller;
using the trained machine learning model to characterize a tissue of the subject during the endoscopic procedure; and
displaying a user interface that communicates a characterization of the tissue of the subject.

2. The method of claim 1, wherein the endoscopic image data comprises video frames captured during the endoscopic procedure.

3. The method of claim 1, wherein the user interface is updated in real-time during the endoscopic procedure.

4. The method of claim 1, wherein preprocessing the endoscopic image data comprises image normalizing to standardize brightness and color values across different endoscopic images.

5. The method of claim 1, wherein the trained machine learning model was generated using a methodology comprising pruning, quantizing, or sparsifying.

6. The method of claim 1, wherein the trained machine learning model comprises a convolutional neural network.

7. The method of claim 1, wherein characterizing the tissue of the subject comprises detecting a presence of a stricture.

8. The method of claim 7, wherein characterizing the tissue of the subject further comprises classifying the detected stricture.

9. The method of claim 1, wherein characterizing the tissue of the subject comprises determining a likelihood of malignancy for observed tissue features.

10. The method of claim 1, wherein the user interface comprises a confidence score for a tissue classification.

11. The method of claim 10, wherein the confidence score is overlaid on a live endoscopic image, and wherein the confidence score is updated in real-time.

12. The method of claim 1, wherein the user interface comprises a visual overlay on the endoscopic image data to highlight a region of interest.

13. The method of claim 1, wherein the trained machine learning model was trained using pathology data.

14. The method of claim 13, wherein the trained machine learning model was trained by comparing the pathology data to annotated training image data.

15. The method of claim 1, further comprising storing the characterization of the tissue for subsequent training of the machine learning model.

16. A method for training a machine learning model to characterize tissue from endoscopic images, comprising:
receiving annotated image data obtained from endoscopic procedures performed on one or more subjects, wherein the annotated image data includes annotations characterizing tissue of the one or more subjects;
using the received annotated image data to train a machine learning model to characterize tissue from endoscopic images;
receiving pathology data that characterizes the tissue of the one or more subjects;
comparing the annotated image data to the pathology data; and
outputting a trained machine learning model based on comparing the annotated image data to the pathology data.

17. The method of claim 31, wherein comparing the annotated image data to the pathology data includes determining whether annotations of the annotated image data include errors.

18. The method of claim 16, wherein the pathology data and annotations of the annotated image data characterize the same tissue.

19. A method for training a machine learning model to characterize tissue from endoscopic images, comprising:
receiving pathology data that characterizes a same tissue that was previously characterized by a trained machine learning model;
comparing the received pathology data to a previous characterization of the same tissue that was made by the trained machine learning model; and
based on the comparing, refining the trained machine learning model.

20. The method of claim 19, wherein refining the trained machine learning model includes adjusting parameters of the machine learning model to improve alignment between the model's previous characterization of the tissue and the pathology data.
